(19) United States
(12) Patent Application Publication
(10) Pub. No.: US 2024/0366579 A1
(43) Pub. Date: Nov. 7, 2024

(54) PHARMACEUTICAL COMPOSITION COMPRISING GPR40 AGONIST AND SGLT-2 INHIBITOR

(71) Applicant: ILDONG PHARMACEUTICAL CO., LTD., Seoul (KR)

(72) Inventors: Jong Min YOON, Hwaseong- si Gyeonggi-do (KR); Don Gil LEE, Hwaseong- si Gyeonggi-do (KR); In Gyu JE, Hwaseong- si Gyeonggi-do (KR); Hong Chul YOON, Hwaseong- si Gyeonggi-do (KR); Joon Tae PARK, Hwaseong- si Gyeonggi-do (KR); Kyung Mi AN, Hwaseong- si Gyeonggi-do (KR); Jung Woo LEE, Hwaseong- si Gyeonggi-do (KR); Haeng Jin SONG, Hwaseong- si Gyeonggi-do (KR); Da Hae HONG, Hwaseong- si Gyeonggi-do (KR)

(21) Appl. No.: 18/557,538

(22) PCT Filed: Apr. 28, 2022
(86) PCT No.: PCT/KR2022/006120

§ 371 Date: Oct. 26, 2023

(30) Foreign Application Priority Data
Apr. 29, 2021 (KR) 10-2021-0055992

(51) Int. Cl. A61K 31/443; A61K 9/00; A61K 9/20; A61K 9/48; A61K 31/7004; A61K 31/7048; A61K 45/06; A61P 3/10

(52) U.S. Cl. CPC A61K 31/443; A61K 31/7004; A61K 31/7048; A61K 45/06; A61P 3/10; A61K 9/0053; A61K 9/20; A61K 9/48

(57) ABSTRACT

Disclosed herein are a pharmaceutical composition for prevention, alleviation, or treatment of any disease selected from the group consisting of type 2 diabetes mellitus, hyperinsulinemia, impaired glucose tolerance disorder, and insulin resistance disorder, wherein the pharmaceutical composition includes a GPR40 agonist and an SGLT2 inhibitor, the GPR40 agonist being a compound represented by Formula 1, a racemate of the compound, an enantiomer of the compound, a diastereomer of the compound, or a pharmaceutically acceptable salt of the compound, the racemate, the enantiomer or the diastereomer, a method of preparing the same, and a method of treating type 2 diabetes mellitus and the like using the same.

### TECHNICAL FIELD

Embodiments of the present disclosure relate to a pharmaceutical composition including a GPR40 agonist and an SGLT-2 inhibitor, a method of preparing the same, and a method of treating type 2 diabetes mellitus and the like using the same.

### BACKGROUND ART

Diabetes mellitus is a metabolic disease resulting from impaired or insufficient insulin secretion and characterized by hyperglycemia, that is, an excess of glucose in the blood, and is a progressive debilitating disorder that causes various microvascular and macrovascular complications and morbidities. Type 2 diabetes mellitus, which is the most common type of diabetes mellitus, is characterized by increased insulin resistance associated with inadequate insulin secretion after a period of compensatory hyperinsulinemia.

Free fatty acids (FFAs) have been demonstrated to influence insulin secretion from β-cells by primarily enhancing glucose-stimulated insulin secretion (GSIS). G-protein-coupled receptors (GPCRs) expressed in β-cells are known to regulate release of insulin in response to changing plasma glucose levels.

GPR40, also known as fatty acid receptor 1 (FFAR1), is a membrane-bound FFA receptor that is preferentially expressed in pancreatic islets, especially β-cells thereof, and mediates medium- to long-chain fatty acid-induced insulin secretion. GPR40 is also expressed in enteroendocrine cells. Activation of GPR40 in enteroendocrine cells promotes secretion of intestinal incretin hormones, for example, GLP-1, GIP, CCK, and PYY. GPR40 modulators not only can promote GSIS through an incretin effect, but also hold promise as a potential combination with a wide variety of antidiabetic drugs. Thus, such GPR40 modulators can contribute to reduction of medical burden of patients with type 2 diabetes through enhanced glycemic control.

In view of these advantages, many pharmaceutical companies have worked on developing a GPR40 agonist for the past several years. However, there are still no commercially available GPR40 agonists. An example of a few tangible results is fasiglifam developed by Takeda Pharmaceutical Co., Ltd. of Japan. Fasiglifam is the first GPR40 agonist compound to enter clinical trials and has proven its hypoglycemic efficacy for patients with type 2 diabetes mellitus. However, development of fasiglifam was discontinued due to concerns about drug-induced hepatotoxicity or drug-induced liver injury (DILI)) during phase 3 clinical trials.

Therefore, in modern societies where the number of patients with metabolic syndrome including diabetes is rapidly increasing, there is a growing need to provide an effective treatment for type 2 diabetes patients using a GPR40 agonist having a function of enhancing glucose-dependent insulin secretion.

As a GPR40 agonist, a compound represented by Formula I, a racemate of the compound, an enantiomer of the compound, a diastereomer of the compound, or a pharmaceutically acceptable salt of the compound, the racemate, the enantiomer or the diastereomer is disclosed in Korean Patent Laid-open Publication No. 10-2018-0069718, the disclosure of which is incorporated by reference in its entirety.

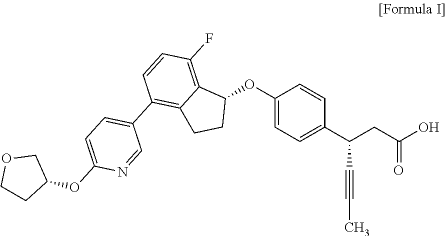 [SMILES: CC#C[C@@H](CC(=O)O)c1ccc(O[C@@H]2CCc3c(-c4ccc(O[C@@H]5CCOC5)nc4)ccc(F)c32)cc1]

Plasma glucose is generally filtered through the renal glomerulus and is actively reabsorbed by the proximal tubule. Sodium-glucose cotransporter 2 (SGLT-2) is considered a major transporter involved in glucose reuptake in the proximal tubule. A selective inhibitor of SGLT-2, which is a sodium-dependent glucose transporter in the kidney, is expected to be able to normalize plasma glucose levels by improving insulin sensitivity and delaying the onset of diabetic complications through enhancement in excretion of glucose in the urine.

### RELATED LITERATURE

### Patent Document

Korean Patent Laid-open Publication No. 10-2018-0069718

### Non-Patent Document

### Disclosure of Invention

### Technical Problem

Embodiments of the present disclosure provide a pharmaceutical composition that can be safely used without causing side effects, such as hypoglycemia, while achieving enhancement in insulin secretion and amelioration of insulin resistance disorder through effective reduction in blood glucose level in diabetic patients.

Embodiments of the present disclosure provide a pharmaceutical composition that can achieve a synergistic effect through combination of two drugs having hypoglycemic effects.

### Solution to Problem

In accordance with one aspect of the present disclosure, there is provided a pharmaceutical composition for prevention, alleviation, or treatment of any disease selected from the group consisting of type 2 diabetes mellitus, hyperinsulinemia, impaired glucose tolerance disorder, and insulin resistance disorder, wherein the pharmaceutical composition includes a GPR40 agonist and an SGLT2 inhibitor, the GPR40 agonist being a compound represented by Formula 1, a racemate of the compound, an enantiomer of the compound, a diastereomer of the compound, or a pharmaceutically acceptable salt of the compound, the racemate, the enantiomer or the diastereomer.

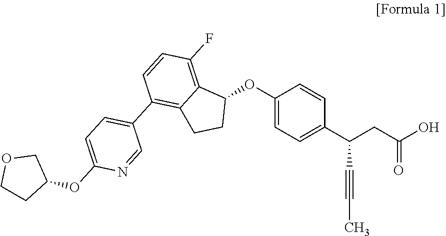 [SMILES: CC#C[C@@H](CC(=O)O)c1ccc(O[C@@H]2CCc3c(-c4ccc(O[C@@H]5CCOC5)nc4)ccc(F)c32)cc1]

In accordance with another aspect of the present disclosure, there is provided a method of preventing, alleviating, or treating any disease selected from the group consisting of type 2 diabetes mellitus, hyperinsulinemia, impaired glucose tolerance, and insulin resistance, wherein the method includes providing a GPR40 agonist and an SGLT2 inhibitor to a patient in need thereof, the GPR40 agonist being a compound represented by Formula 1, a racemate of the compound, an enantiomer of the compound, a diastereomer of the compound, or a pharmaceutically acceptable salt of the compound, the racemate, the enantiomer, or the diastereomer.

In accordance with a further aspect of the present disclosure, there is provided a method of preparing a pharmaceutical composition for prevention, alleviation, or treatment of any disease selected from the group consisting of type 2 diabetes mellitus, hyperinsulinemia, impaired glucose tolerance, and insulin resistance, wherein the method includes formulating a GPR40 agonist and an SGLT2 inhibitor in a single dosage form or separate dosage forms, the GPR40 agonist being a compound represented by Formula 1, a racemate of the compound, an enantiomer of the compound, a diastereomer of the compound, or a pharmaceutically acceptable salt of the compound, the racemate, the enantiomer, or the diastereomer.

### Advantageous Effects of Invention

The pharmaceutical composition according to the present disclosure can be safely used without causing side effects such as hypoglycemia through combined use of the GPR40 agonist and the SGLT2 inhibitor resulting in greater effects than separate use thereof and thus allowing reduction in dosage, while achieving enhancement in insulin secretion and amelioration of insulin resistance disorder through effective reduction in blood glucose level in diabetic patients.

In addition, the pharmaceutical composition according to the present disclosure can effectively reduce blood glucose levels in diabetic patients through synergistic hypoglycemic effects of the GPR40 agonist and the SGLT2 inhibitor.

### BRIEF DESCRIPTION OF DRAWINGS

FIG. 1A shows results of measurement of changes in blood glucose level from 0 to 120 min after combined administration of a compound represented by Formula 1 and dapagliflozin, FIG. 1B shows results of measurement of the area under the blood glucose curve (AUC) from 0 to 60 min after the combined administration, and FIG. 1C shows results of measurement of AUC from 0 to 120 min after the combined administration.

FIG. 2A shows results of measurement of changes in blood glucose level from 0 to 120 min after combined administration of the compound represented by Formula 1 and empagliflozin, FIG. 2B shows results of measurement of the area under the blood glucose curve (AUC) from 0 to 60 min after the combined administration, and FIG. 2C shows results of measurement of AUC from 0 to 120 min after the combined administration.

### MODE FOR THE INVENTION

One aspect of the present disclosure relates to a pharmaceutical composition for prevention, amelioration, or treatment of any disease selected from the group consisting of type 2 diabetes mellitus, impaired fasting glucose, hyperglycemia, impaired glucose tolerance disorder, and insulin resistance disorder, wherein the pharmaceutical composition includes: a compound represented by Formula 1, a racemate of the compound, an enantiomer of the compound, a diastereomer of the compound, or a pharmaceutically acceptable salt of the compound, the racemate, the enantiomer, the

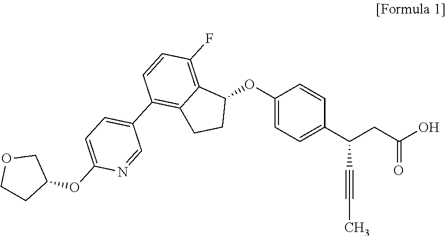 [SMILES: CC#C[C@@H](CC(=O)O)c1ccc(O[C@@H]2CCc3c(-c4ccc(O[C@@H]5CCOC5)nc4)ccc(F)c32)cc1]

The compound represented by Formula 1 is a compound disclosed as Example 5 in Korean Patent Laid-open Publication No. 10-2018-0069718, the general formula of which is (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid.

The compound has an ability to activate GPR40, can be administered orally, and has a mechanism of inducing glucose-dependent insulin secretion. Thus, the compound is very effective in lowering blood glucose to a normal level without causing side effects such as hypoglycemia.

The compound represented by Formula 1 may be used in the form of a racemate, an enantiomer, a diastereomer or a pharmaceutically acceptable salt thereof. Specifically, it is preferred in terms of solubility or stability that the compound be used in the form of a free acid of an S-isomer thereof.

Furthermore, the compound has a preventive and/or therapeutic effect on obesity and hypertension through a direct or indirect mechanism in which GPR40 is involved based on hypoglycemic action thereof. Accordingly, another aspect of the present disclosure relates to novel pharmaceutical use of the pharmaceutical composition for prevention, amelioration, or treatment of any metabolic disease selected from the group consisting of hyperlipidemia, hypertriglyceridemia, hypercholesterolemia, and dyslipidemia.

The inventors of the present disclosure found that use of the compound represented by Formula 1 in combination with a sodium-glucose cotransporter 2 (SGLT2) inhibitor can exhibit synergistic hypoglycemic action that far exceeds the simple sum of hypoglycemic effects of these two drugs acting independently, and thus can significantly reduce side effects, as compared with when the two drugs are used alone to have the same level of hypoglycemic effects.

Examples of an SGLT2 inhibitor that can be administered along with the compound represented by Formula 1 may include canagliflozin, dapagliflozin, empagliflozin, bexagliflozin, ertugliflozin, remogliflozin, tofogliflozin, luseogliflozin, ipragliflozin, and sotagliflozin. Specifically, the SGLT2 inhibitor may be dapagliflozin, canagliflozin, ertugliflozin, or empagliflozin. More specifically, the SGLT2 inhibitor may be dapagliflozin or empagliflozin. The SGLT2 inhibitor may be in the form of a free acid thereof or in the form of a pharmaceutically acceptable salt, ester, or solvate thereof.

Although the compound represented by Formula 1 and the SGLT2 inhibitor constituting the composition may be contained together in one formulation, it should be understood that the present disclosure is not limited thereto and the compound represented by Formula 1 and the SGLT2 inhibitor may be administered separately or sequentially. That is, the compound represented by Formula 1 and the SGLT2 inhibitor may be present in separate dosage forms to be separately or sequentially administered to a patient. However, it is preferred in terms of medication convenience and compliance that the compound represented by Formula 1 and the SGLT2 inhibitor be contained together in one formulation.

The composition may be administered once or multiple times to a subject in need thereof, and may be administered at a dose capable of obtaining the maximum effect with minimum medication without side effects. Specifically, an effective dose of the pharmaceutical composition according to the present disclosure may vary depending on a patient's age, sex, condition, weight, absorption of active ingredients, disease type, and other drugs.

A weight ratio of the compound represented by Formula 1 to the SGLT2 inhibitor may be in the range of 0.2:9.8 to 9:1, specifically 1:9 to 8:2, and more specifically 2:8 to 7:3. Within this range, each of the drugs can exert hypoglycemic effects through a pharmacological mechanism specific thereto and a synergistic effect in glycemic control can be produced through the different pharmacological mechanisms of the two drugs.

The compound represented by Formula 1 may be present in an amount of 0.5% by weight (wt %) to 20 wt %, specifically 0.5 wt % to 10 wt %, based on the total weight of the pharmaceutical composition, and the SGLT2 inhibitor may be present in an amount of 0.5 wt % to 20 wt %, specifically 0.5 wt % to 10 wt %, based on the total weight of the pharmaceutical composition.

A daily dosage of the composition may be in the range of about 0.0001 mg/kg to 100 mg/kg, specifically 0.1 mg/kg to 50 mg/kg, and may be given as a single dose or in divided doses, without being limited thereto. When the daily dosage of the pharmaceutical composition is in the range of 0.1 mg/kg to 50 mg/kg, it is possible to ensure proper and safe use of the pharmaceutical composition without side effects such as hypoglycemia while achieving enhancement in insulin secretion and amelioration of insulin resistance disorder in diabetic patients through effective reduction in blood glucose level. A dosage of the compound represented by Formula 1 may be in the range of 0.01 mg/day to 100 mg/day, specifically 0.1 mg/day to 50 mg/day, and a dosage of the SGLT2 inhibitor may be in the range of 0.01 mg/day to 100 mg/day, specifically 0.1 mg/day to 50 mg/day.

The pharmaceutical composition may further include a pharmaceutically acceptable carrier, excipient, or diluent, apart from the compound represented by Formula 1 and the SGLT2 inhibitor.

The pharmaceutically acceptable carrier, excipient, or diluent may include lactose, dextrose, sucrose, sorbitol, mannitol, xylitol, erythritol, maltitol, starch, acacia gum, calcium phosphate, alginate, gelatin, calcium silicate, methyl cellulose, cellulose, microcrystalline cellulose, polyvinylpyrrolidone, water, methyl hydroxybenzoate, propyl hydroxybenzoate, talc, magnesium stearate, and mineral oil, without being limited thereto. The pharmaceutically acceptable carrier, excipient, or diluents may be present in an amount of 1 wt % to 85 wt % or 5 wt % to 75 wt % based on the total solid weight of the pharmaceutical composition.

In formulation of the pharmaceutical composition, a diluent or excipient commonly used in the art, such as a filler, a binder, a bulking agent, a wetting agent, a disintegrant, and a surfactant, may be used.

The pharmaceutical composition according to the present disclosure may be administered to a subject by various routes. The pharmaceutical composition may be administered by any suitable route known in the art. For example, the pharmaceutical composition may be administered orally, intranasaly, transbronchially, intra-arterially, intravenously, hypodermically, intramuscularly, or intraperitoneally. Specifically, the pharmaceutical composition may be administered in a solid oral dosage form, such as a tablet or a capsule. More specifically, the pharmaceutical composition may be prepared in the form of a monolayer tablet, a bilayer tablet, or a core-shell type bilayer tablet.

The pharmaceutical composition according to the present disclosure may further include another antidiabetic agent or hypoglycemic agent without affecting the synergistic effect of the two drugs of the present disclosure, that is, the compound represented by Formula 1 and the SGLT2 inhibitor.

For example, the antidiabetic agent or hypoglycemic agent may include: a biguanide drug selected from the group consisting of metformin, buformin, and phenformin; an insulin sensitizer selected from the group consisting of troglitazone, ciglitazone, rosiglitazone, pioglitazone, and englitazone; a DPP4 inhibitor selected from the group consisting of sitagliptin, linagliptin, vildagliptin, gemigliptin, saxagliptin, alogliptin, teneligliptin, anagliptin, and evogliptin; a glucagon-like peptide (GLP) 1 agonist selected from the group consisting of exenatide, lixisenatide, liraglutide, albiglutide, and dulaglutide; an insulin secretagogue selected from the group consisting of glycylamide, glycentide, glypentide, glipizide, glibenclamide, gliclazide, glimepiride, tolazamide, tolbutamide, acetohexamide, carbutamide, chlorpropamide, glibornuride, gliquidone, glycoamide, glisoxepide, and glyclopiamide; and an α-glucosidase inhibitor selected from the group consisting of acarbose, voglibose, emiglitate, and miglitol.

A further aspect of the present disclosure relates to a method for prevention, alleviation, or treatment of any disease selected from the group consisting of type 2 diabetes mellitus, hyperinsulinemia, impaired glucose tolerance disorder, and insulin resistance disorder, wherein the method includes providing a GPR40 agonist and an SGLT2 inhibitor to a patient in need thereof, the GPR40 agonist being the compound represented by Formula 1 or a racemate, enantiomer, diastereomer or pharmaceutically acceptable salt thereof. In the method, the compound represented by Formula 1 and the SGLT2 inhibitor may be administered in one formulation, or may be administered separately or sequentially to enhance therapeutic effects thereof. Accordingly, in the method, the compound represented by Formula 1 and the SGLT2 inhibitor may also be present in separate dosage forms to be sequentially or separately administered to a patient.

For details of the dosage, administration method, and administration route of each of the drugs in the treatment method, refer to the aforementioned aspect of the present disclosure.

Yet another aspect of the present disclosure relates to a method of preparing a pharmaceutical composition for prevention, alleviation, or treatment of any disease selected from the group consisting of type 2 diabetes mellitus, hyperinsulinemia, impaired glucose tolerance disorder, and insulin resistance disorder, wherein the method includes formulating a GPR40 agonist and an SGLT2 inhibitor along with or separately from a pharmaceutically acceptable excipient, carrier, or diluent, the GPR40 agonist being the compound represented by Formula 1, a racemate of the compound, an enantiomer of the compound, a diastereomer of the compound, or a pharmaceutically acceptable salt of the compound, the racemate, the enantiomer, the diastereomer.

For details of the dosage, administration method, and administration of each of the drugs in the preparation method, refer to the aforementioned aspect of the disclosure.

Next, the present disclosure will be described in more detail with reference to some examples. However, it should be noted that these examples are provided for illustration only and are not to be construed in any way as limiting the present disclosure.

### EXAMPLE

### Example 1: Synergistic Effects of Combined Use of the Compound Represented by Formula 1 and Dapagliflozin on Reduction in Blood Glucose Level

In this experiment, the compound represented by Formula 1 was prepared by the method described in Example 5 of Korean Patent Laid-Open Publication No. 10-2018-0069718 and dapagliflozin (HY-10450, MedChemExpress Co., Ltd.) was used. As a control group, a vehicle (0.5% carboxymethyl cellulose (CMC)) was used.

After at least 1 week of acclimatization of 8 to 10 week-old male Sprague-Dawley (SD) rats, an oral glucose tolerance test (OGTT) was performed using healthy rats.

After 16 hours of fasting, the rats were randomly divided into groups each having 5 rats similar in body weight and blood glucose level, followed by administration of the vehicle (0.5% carboxymethyl cellulose (CMC)), 0.3 mg/kg of the compound represented by Formula 1 alone, 0.1 mg/kg of dapagliflozin alone, or a combination of 0.3 mg/kg of the compound represented by Formula 1 and 0.1 mg/kg of dapagliflozin. One hour after administration of each of the drugs, glucose (2 g/kg) was intraperitoneally injected at a dose of 10 ml/kg. Blood glucose level was measured 60 minutes before glucose injection and 0, 15, 30, 60, and 120 minutes after glucose injection through puncture of the caudal vein using a blood glucose meter (Gluco DR. Almedicus. Co. Ltd.). Results were expressed by a reduction rate (%) in area under the blood glucose curve (AUC) relative to administration of the vehicle.

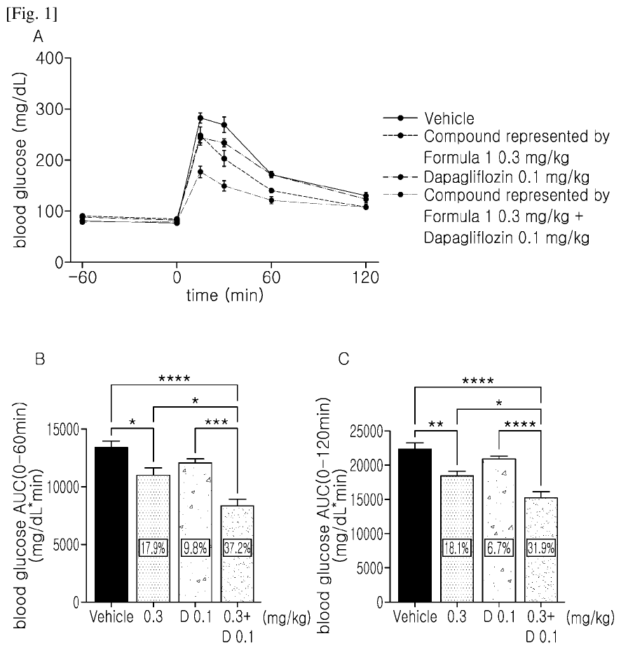

Results of measuring AUC from 0 to 60 min and from 0 to 120 min after glucose injection according to the above method are shown in Table 1 and FIG. 1.

TABLE 1

| | Compound | Dapagli- | Simple sum of | Combination of |
|---|---|---|---|---|
| | of Formula 1 | flozin | Compound of | compound of |
| | (0.3 | (0.1 | Formula 1 and | Formula 1 and |
| | mg/kg) | mg/kg) | dapagliflozin | dapagliflozin |
| AUC | 17.9% | 9.8% | 27.7% | 37.2% |
| (0-60 min) | | | | (>27.7%) |
| AUC | 18.1% | 6.7% | 24.8% | 31.9% |
| (0-120 min) | | | | (>24.8%) |

### Example 2: Synergistic Effects of Combined Use of the Compound Represented by Formula 1 and Empagliflozin on Reduction in Blood Glucose Level

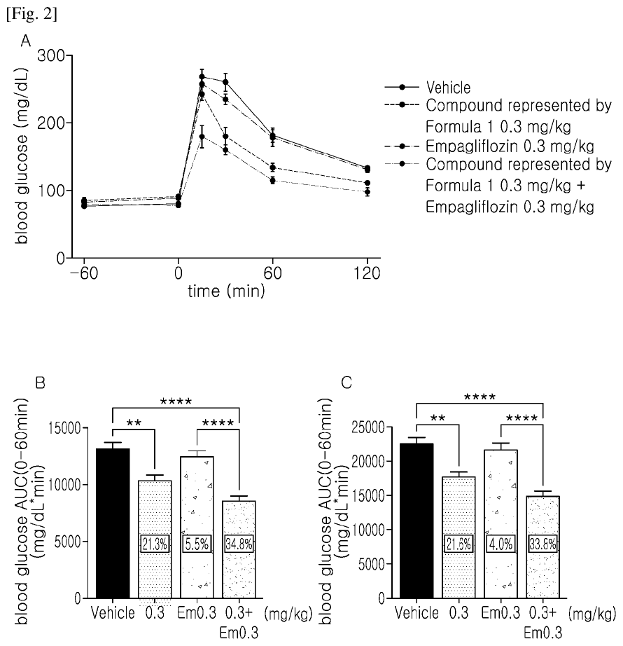

An experiment was conducted in the same manner as in Example 1 except that 0.3 mg/kg of empagliflozin (HY-15409, MedChemExpress Co., Ltd.) was used instead of 0.1 mg/kg of dapagliflozin. Results are shown in Table 2 and FIG. 2.

TABLE 2

| | Compound | | Simple sum of | Combination of |
|---|---|---|---|---|
| | represented by | Empagli- | Compound | compound |
| | Formula 1 | flozin | represented by | represented by |
| | (0.3 | (0.3 | Formula 1 and | Formula 1 and |
| | mg/kg) | mg/kg) | empagliflozin | empagliflozin |
| AUC | 21.3% | 5.5% | 26.8% | 34.8% |
| (0-60 | | | | (>26.8%) |
| min) | | | | |
| AUC | 21.6% | 4.0% | 25.6% | 33.8% |
| (0-120 | | | | (>25.6%) |
| min) | | | | |

The results show that combined administration of the compound represented by Formula 1 and the SGLT2 inhibitor (dapagliflozin or empagliflozin) can produce a synergistic effect in reducing blood glucose levels, as compared with administration of the compound represented by Formula 1 alone or administration of dapagliflozin or empagliflozin alone.

1. A method for treating a disease selected from the group consisting of type 2 diabetes mellitus, hyperinsulinemia, impaired glucose tolerance disorder, and insulin resistance disorder, wherein the method comprises administering a pharmaceutical composition comprising a compound represented by Formula 1, a racemate of the compound, an enantiomer of the compound, a diastereomer of the compound, or a pharmaceutically acceptable salt of the compound, the racemate, the enantiomer, or the diastereomer and an SGLT2 inhibitor, to a subject in need thereof.

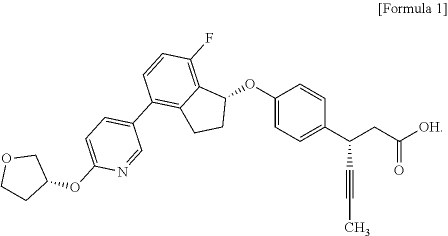 [SMILES: CC#C[C@@H](CC(=O)O)c1ccc(O[C@@H]2CCc3c(-c4ccc(O[C@@H]5CCOC5)nc4)ccc(F)c32)cc1]

2. The method according to claim 1, wherein the SGLT2 inhibitor is selected from the group consisting of canagliflozin, dapagliflozin, empagliflozin, bexagliflozin, ertugliflozin, remogliflozin, tofogliflozin, luseogliflozin, ipragliflozin, and sotagliflozin.

3. The method according to claim 2, wherein the SGLT2 inhibitor is dapagliflozin or empagliflozin.

4. The method according to claim 1, wherein the pharmaceutical composition is administered in a solid oral dosage form.

5. The method according to claim 4, wherein the solid oral dosage form is a tablet or capsule.

6. The method according to claim 1, wherein the pharmaceutical composition further comprises a pharmaceutically acceptable carrier, excipient, or diluent.

7. The method according to claim 1, wherein a weight ratio of the compound represented by Formula 1 to the SGLT2 inhibitor ranges from 0.2:9.8 to 9:1.

8. The method according to claim 1, wherein the compound represented by Formula 1 is in the form of a free acid thereof.

9. The method according to claim 1, wherein the method further comprises administering one or more drugs selected from the group consisting of:
a biguanide drug selected from the group consisting of metformin, buformin, and phenformin;
an insulin sensitizer selected from the group consisting of troglitazone, ciglitazone, rosiglitazone, pioglitazone, and englitazone;
a DPP4 inhibitor selected from the group consisting of sitagliptin, linagliptin, vildagliptin, gemigliptin, saxagliptin, alogliptin, teneligliptin, anagliptin, and evogliptin;
a glucagon-like peptide (GLP) 1 agonist selected from the group consisting of exenatide, lixisenatide, liraglutide, albiglutide, and dulaglutide;
an insulin secretagogue selected from the group consisting of glycylamide, glycentide, glypentide, glipizide, glibenclamide, gliclazide, glimepiride, tolazamide, tolbutamide, acetohexamide, carbutamide, chlorpropamide, glibornuride, gliquidone, glycoamide, glisoxepide, and glyclopiamide; and
an α-glucosidase inhibitor selected from the group consisting of acarbose, voglibose, emiglitate, and miglitol.

10. A method of preparing a pharmaceutical composition for prevention, alleviation, or treatment of any disease selected from the group consisting of type 2 diabetes mellitus, hyperinsulinemia, impaired glucose tolerance disorder, and insulin resistance disorder, the method comprising formulating a GPR40 agonist and an SGLT2 inhibitor along with or separately from a pharmaceutically acceptable excipient, carrier, or diluent, wherein the GPR40 agonist is a compound represented by Formula 1, a racemate of the compound, an enantiomer of the compound, a diastereomer of the compound, or a pharmaceutically acceptable salt of the compound, the racemate, the enantiomer, or the diastereomer 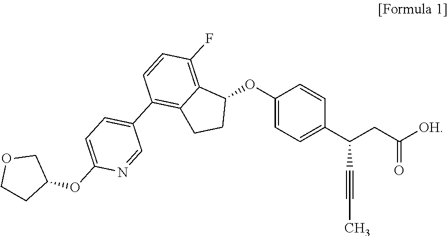 [SMILES: CC#C[C@@H](CC(=O)O)c1ccc(O[C@@H]2CCc3c(-c4ccc(O[C@@H]5CCOC5)nc4)ccc(F)c32)cc1]
